(19) United States
(12) Patent Application Publication
(10) Pub. No.: US 2022/0409496 A1
(43) Pub. Date: Dec. 29, 2022

(54) ANTI-UV COSMETIC COMPOSITION

(71) Applicant: COATEX, Genay (FR)

(72) Inventors: Clementine CHAMPAGNE, Caluire-et-Cuire (FR); Helene PERNOT, Lyon (FR)

(73) Assignee: COATEX, Genay (FR)

(21) Appl. No.: 17/764,432

(22) PCT Filed: Sep. 28, 2020
(86) PCT No.: PCT/FR2020/000245

§ 371 Date: Mar. 28, 2022

(30) Foreign Application Priority Data
Sep. 30, 2019 (FR) 1910779

(51) Int. Cl. A61K 8/06; A61K 8/37; A61K 8/29; A61K 8/25; A61K 8/891; A61K 8/67; A61K 8/40; A61K 8/35; A61Q 17/04

(52) U.S. Cl. CPC A61K 8/062; A61K 8/37; A61K 8/29; A61K 8/25; A61K 8/891; A61K 8/678; A61K 8/40; A61K 8/35; A61Q 17/04

(57) ABSTRACT

Anti-UV cosmetic compositions in the form of an emulsion may include an emulsifying polymer which is prepared by polymerizing an anionic monomer and a C1-C7 ester of an unsaturated carboxylic acid. Such anti-UV cosmetic compositions may be prepared in the absence of a surfactant. The preparation and use of such anti-UV cosmetic compositions may provide anti-UV cosmetic composition having dual protection for the skin: protection against UV rays and limiting the penetration of UV filters into the skin.

The invention relates to a UV-stabilizing cosmetic composition in the form of an emulsion comprising an emulsifying polymer that is prepared by polymerisation of an anionic monomer and of a C₁-C₇ ester of an unsaturated carboxylic acid. The UV-stabilizing cosmetic composition according to the invention is prepared in the absence of any surface-active compound. The invention also relates to the preparation and use of this UV-stabilizing cosmetic composition. The UV-stabilizing cosmetic composition according to the invention provides dual skin protection: it protects against UV radiation and limits the penetration of the UV-stabilizing filters into the skin.

The invention thus provides a photoprotective composition that makes it possible to reduce the risks of skin sensitization, particularly in the areas of the skin that are naturally exposed to light or that frequently undergo overexposure to light. These areas of the skin usually require the use of photoprotective products to reduce or avoid the harmful effects of solar radiation.

Photoprotective compositions generally use substances such as UV-stabilizing agents that may have some skin sensitizing properties. Most often, sensitization results from the UV-stabilizing agent passing through the skin barrier.

In order to limit or avoid such sensitization, penetration of the UV-stabilizing agents should, insofar as possible, be avoided or limited, particularly penetration through the skin barrier. The photoprotective compositions used should therefore allow the UV-stabilizing filters to be retained in skin areas less likely to react with the UV-stabilizing agent. There is therefore a need for improved UV-stabilizing cosmetic compositions that make it possible to retain the UV-stabilizing filters in the cutaneous areas irrespective of the viscosity of the composition or irrespective of the suspensive properties of the composition.

There are known cosmetic compositions, particularly cosmetic compositions in the form of oil-in-water emulsions in which a lipophilic phase is dispersed in a hydrophilic phase.

These known cosmetic compositions in the form of emulsions usually require the use of a surface-active agent in their preparation. Such emulsions must be stable and must have, for each use, a specific texture that makes them effective and pleasant to the touch. These cosmetic compositions must also be easy to apply. Generally speaking, cosmetic compositions comprise a great many ingredients. The reduction in the number of ingredients while retaining the properties of the cosmetic compositions when used, but also during their preparation, is constantly sought.

All of these properties are particularly desirable for cosmetic sunscreen compositions. This is also true for the cosmetic compositions used in makeup, for emollient cosmetic compositions and for foundation compositions.

Document WO 2019122784 describes a method for preparing an emulsion by dispersion of a lipophilic compound in a continuous hydrophilic phase. This emulsion does not comprise a UV-stabilizing agent. Document WO 2014139901 describes a sunscreen composition comprising a photoprotection agent combined with a thickening homopolymer and a styrenic copolymer. Document EP 1093798 discloses cosmetic or dermatological emulsions comprising an organic UV-stabilizing filter in micronized form. Document FR 2972370 describes a method for preparing cosmetic compositions. Document WO 2019185999 describes a method for preparing a water-resistant cosmetic composition but which does not include a UV-stabilizing agent.

There is thus a need for improved UV-stabilizing cosmetic compositions as well as for methods of preparing or using such UV-stabilizing cosmetic compositions that are also improved.

The UV-stabilizing cosmetic composition according to the invention provides a solution to all or part of the problems of the cosmetic compositions in the prior art.

Thus, the invention provides a UV-stabilizing cosmetic composition in the form of an emulsion prepared in the absence of any surface-active compounds, and comprising:
from 1 to 75% by weight of the composition, of particles of a lipophilic phase comprising at least one lipophilic compound and at least one UV-stabilizing agent chosen among an organic sunscreen, mineral particles and combinations thereof, dispersed in:
from 25 to 99% by weight of the composition, of a continuous hydrophilic phase comprising:
   from 90 to 99.6% by weight of the hydrophilic phase, of at least one hydrophilic compound and
   from 0.4 to 10% by weight of the hydrophilic phase, of at least one emulsifying polymer (P) prepared by at least one polymerisation reaction:
   (a1) of at least one anionic monomer comprising at least one polymerisable olefinic unsaturation and at least one carboxylic acid group or of one of its salts and
   (a2) of at least one C₁-C₇ ester of a compound derived from an acid chosen among acrylic acid, methacrylic acid, maleic acid, maleic anhydride, itaconic acid and crotonic acid.

In the composition according to the invention, the amounts of lipophilic phase and hydrophilic phase can vary, in particular according to the desired texture or according to the final use of the composition.

Preferably, the composition according to the invention comprises from 1 to 70% by weight or from 5 to 70% by weight of dispersed lipophilic phase relative to the total amount by weight of continuous hydrophilic phase and of dispersed lipophilic phase. Also preferably, the composition according to the invention comprises from 1 to 65% by weight or from

5 to 65% by weight of dispersed lipophilic phase relative to the total amount by weight of continuous hydrophilic phase and of dispersed lipophilic phase.

More preferably, the composition according to the invention comprises from 1 to 60% by weight or from 5 to 60% by weight of dispersed lipophilic phase relative to the total amount by weight of continuous hydrophilic phase and of dispersed lipophilic phase.

The UV-stabilizing cosmetic composition according to the invention comprises at least one lipophilic compound in the form of particles to form the lipophilic phase. Preferably, the lipophilic compound is a cosmetic compound.

Preferably according to the invention, the lipophilic phase comprises at least one lipophilic compound chosen among the fatty acids, fatty alcohols, esters, butters, waxes (for example beeswaxes), oils, preferably an oil chosen among the mineral oils (for example paraffin oil, vaseline oil, mineral oils with a boiling point ranging from 300 to 400° C.), animal oils (for example squalenes, squalane, perhydrosqualene), vegetable oils (for example sweet almond oil, calophyllum oil, palm oil, apricot kernel oil, avocado oil, jojoba oil, olive oil, castor oil, grain germ oils, the liquid fraction of shea butter), unsaponifiable compounds derived from natural oils, synthetic oils (for example hydrogenated polyisobutene, fatty acid esters such as purcellin oil, butyl myristate, isopropyl myristate, cetyl myristate, isopropyl palmitate, butyl stearate, hexadecyl stearate, isopropyl stearate, octyl stearate, isocetyl stearate, decyl oleate, hexyl laurate, propylene glycol dicaprylate, esters derived from lanolic acid such as disopropyl lanolate, isocetyl lanolate, acetyl glycerides, octanate alcohols, octanate polyalcohols, decanoate alcohols, decanoate polyalcohols in particular octanoate glycols, octanoate glycerols, decanoate glycols, decanoate glycerols, ricinoleate alcohols, ricinoleate polyalcohols), terpenes, polyterpenes, phytosterol, silicone oils (for example cyclomethicones, low-molecular weight polydimethylsiloxanes or silicone oils, high-molecular weight polydimethylsiloxanes or silicone gums, polymethylsiloxanes, dimethiconols, phenyl polydimethylsiloxanes, low-molecular weight siloxanols, high-molecular weight siloxanols, trimethylsiloxy silicates), fluorinated oils (for example fluorinated perfluoroethers and fluorinated silicones).

The UV-stabilizing cosmetic composition according to the invention comprises at least one UV-stabilizing agent in the lipophilic phase. Preferably according to the invention, the UV-stabilizing agent is chosen among:
titanium dioxide particles, zinc oxide particles, coated mineral particles, in particular coated with a hydrophobic compound, for example stearic acid, in particular titanium dioxide particles coated with a hydrophobic compound, for example stearic acid, and combinations thereof,
lipophilic UV-stabilizing agents, preferably chosen among avobenzone, ethylhexyltriazone, benzophenone-3, octocrylene, benzophenone-2, benzophenone-4, ethyl hexyl salicylate, 4-methyl benzylidene camphor, octyl-N,N-di methyl PABA, ethylhexyl dimethyl PABA, ethylhexyl methoxycinnamate, isoamyl methoxycinnamate, butyl methoxydibenzoylmethane, diethylamino hydroxybenzoyl hexyl benzoate, bis-ethylhexyloxyphenol methoxyphenyl triazine, drometrizole trisiloxane, methylene bis-benzotriazolyl tetramethylbutylphenol, diethylhexyl butamido triazone, ethylhexyl triazone, isoamyl p-methoxycinnamate, poly silicone-15, tris biphenyl triazine, homomenthyl salicylate, PEG-25 PABA, active lipophilic cosmetic molecules, active water-soluble cosmetic molecules, and combinations thereof,
combinations thereof, particularly combinations of titanium dioxide particles and of at least one organic sun filter.

Particularly preferably according to the invention, the UV-stabilizing agent combines titanium dioxide particles and at least one organic sun filter chosen among lipophilic UV-stabilizing agents, preferably chosen among avobenzone, ethylhexyltriazone, benzophenone-3, octocrylene, benzophenone-2, benzophenone-4, ethylhexylsalicylate, 4-methylbenzylidene camphor, octyl-N,N-dimethyl PABA, ethylhexyl dimethyl PABA, ethylhexyl methoxycinnamate, isoamyl methoxycinnamate, butyl methoxydibenzoylmethane, diethylamino hydroxybenzoyl hexyl benzoate, bis-ethyl hexyl oxyphenol methoxyphenyl triazine, drometrizole tri si 1 oxane, methylene bis-benzotriazolyl tetramethylbutylphenol, diethylhexyl butamido triazone, ethylhexyl triazone, isoamyl p-methoxy cinnamate, poly silicone-15, tris biphenyl triazine, homomenthyl salicylate, PEG-25 PABA, active lipophilic cosmetic molecules, active water-soluble cosmetic molecules. Also particularly preferably according to the invention, the UV-stabilizing agent combines several of these organic sun filters.

In general, the composition according to the invention comprises an amount of UV-stabilizing agent ranging from 0.1 to 10% by mass and, more preferably, from 1 to 5% by mass, relative to the total mass of the composition.

In the composition according to the invention, the lipophilic phase is dispersed in the hydrophilic phase which comprises at least one hydrophilic compound. Preferably according to the invention, the hydrophilic compound is chosen among water alone or in combination with at least one compound chosen among hydrophilic UV-stabilizing agents, glycerol, polyglycerols, glycols, for example propylene glycol, butylene glycol, moisteners, for example moisteners for cosmetic compositions, sugar derivatives, for example xylytol, maltilol. According to the invention, the hydrophilic phase can be different from glycerol.

Preferably, the composition according to the invention has a pH greater than 4, more preferentially greater than 5 and much more preferentially greater than 6 or greater than 6.5.

Also preferably, the composition according to the invention has a pH less than 13, more preferentially less than 12 and much more preferentially less than 11.

Also preferably, the composition according to the invention has a pH ranging from 4 to 13 or from 4 to 12 or from 4 to 11, more preferentially ranging from 5 to 13 or from 5 to 12 or from 5 to 11, and much more preferentially ranging from 6 to 13 or from 6 to 12 or from 6 to 11, also more preferentially ranging from 6.5 to 13 or from 6.5 to 12 or from 6.5 to 11.

Essentially according to the invention, the hydrophilic continuous phase comprises at least one hydrophilic compound and at least one emulsifying polymer (P) prepared by at least one polymerisation reaction of monomers (a1) and (a2). Although it has an emulsifying character, the polymer (P) according to the invention is not a surface-active compound. Indeed, the copolymer (P) is not an amphiphilic compound.

The polymer (P) used according to the invention is known as such. It can be prepared by known methods, in particular by a radical polymerisation reaction, for example a polymerisation reaction in an emulsion, in a dispersion or in a solution. The polymerisation can be carried out in a solvent in the presence of at least one initiator compound. As examples of initiator compounds, at least one compound can be used chosen among the azoic initiator compounds (for example azobisisobutyronitrile), a peroxide compound, preferably hydrogen peroxide, benzoyl peroxide, benzoyl hydroperoxide and mixtures thereof. Alkaline metal persulphates can also be mentioned, particularly sodium persulphate and potassium persulphate, ammonium persulphate, partially water-soluble peroxides, particularly succinic peracid, t-butyl hydroperoxide, cumyl hydroperoxide, persulphates combined with a copper ion, a ferrous ion, a sulphite ion or a bisulphite ion and mixtures thereof.

Aside from the various monomers, the method for preparing the polymer (P) generally uses at least one chain transfer agent, preferably chosen among mercaptan compounds, in particular mercaptan compounds comprising at least 4 carbon atoms such as butyl mercaptan, n-octyl mercaptan, n-dodecyl mercaptan, tert-dodecyl mercaptan, iso-octyl 3-mercaptopropionate.

According to the invention, the compound initiator or radical generator can therefore be combined with at least one controlled radical polymerisation transfer agent, in particular a RAFT transfer agent (reversible addition-fragmentation chain transfer or controlled radical polymerisation by reversible chain transfer by addition-fragmentation). Preferably, the reaction is a radical polymerisation reaction in an emulsion.

Preferably according to the invention, monomer (a1) is chosen among acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof. Much more preferentially, monomer (a1) is chosen among acrylic acid, an acrylic acid salt, methacrylic acid, a methacrylic acid salt and combinations thereof.

Preferably according to the invention, monomer (a2) is a C₁-C₆ ester or a C₁-C₄ ester. Also preferably according to the invention, monomer (a2) is a C₁-C₇ acrylic acid ester or a C₁-C₇ methacrylic acid ester, more preferentially chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof. More preferentially, monomer (a2) is chosen among ethyl acrylate, butyl acrylate, methyl methacrylate and combinations thereof.

Particularly preferably according to the invention, monomer (a1) is chosen among acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt and combinations thereof, much more preferentially acrylic acid or methacrylic acid; and monomer (a2) is a C₁-C₆ ester or a C₁-C₄ ester or is a C₁-C₇ acrylic acid ester or a C₁-C₇ methacrylic acid ester, preferably chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof, more preferentially ethyl acrylate, butyl acrylate, methyl methacrylate and combinations thereof.

Also preferably, the polymerisation reaction uses:
from 20 to 75 mol % or from 25 to 70 mol %, preferably from 30 to 70 mol % or from 35 to 65 mol %, of monomer (a1) and
from 25 to 80 mol % or from 30 to 75 mol %, preferably from 30 to 70 mol % or from 35 to 65 mol %, of monomer (a2),
relative to the total molar amount of monomers (a1) and (a2).

In addition to monomers (a1) and (a2), the polymer (P) can be prepared from other monomers. Thus, the polymer (P) can be prepared by a polymerisation reaction that also uses:

(a3) at least one compound chosen among 2-acrylamido-2-methylpropane sulphonic acid, ethoxymethacrylate sulphonic acid, sodium methallyl sulphonate, styrene sulphonate hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, their salts and combinations thereof or

(a4) at least one compound chosen among hydroxyethyl acrylate, hydroxypropyl acrylate, hydroxyethylhexyl acrylate, hydroxyethyl methacrylate, hydroxypropyl methacrylate, hydroxyethylhexyl methacrylate or

(a5) at least one cross-linking monomer or at least one monomer comprising at least two olefinic unsaturations or

(a6) at least one associative monomer.

Also advantageously according to the invention, the polymerisation reaction can use less than 20 mol %, preferably from 0.2 to 20 mol %, particularly from 0.5 to 10 mol %, of monomer (a3) relative to the total molar amount of monomers.

Also advantageously, the polymerisation reaction can use less than 20 mol %, preferably from 0.2 to 20 mol %, particularly from 0.5 to 10 mol %, of monomer (a4) relative to the total molar amount of monomers.

According to the invention, monomer (a5) can be chosen among:
a compound of formula (I):

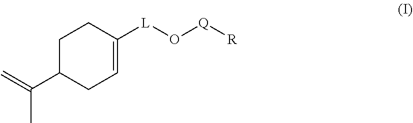

wherein:
   L represents CH₂, CH₂ monoalkoxylate or CH₂ polyalkoxylate, preferably a CH₂ monoethoxylate group or a CH₂ polyethoxylate group,
   Q represents a direct bond or C(O),
   R represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, Q³OQ⁴OC(O)C(CH₃)═CH₂ or Q³OQ⁴OC(O)C(H)═CH₂,
   Q³ represents a difunctional residue of an asymmetric diisocyanate compound, preferably chosen among tolyl-1,3-diisocyanate (TDI) and isophorone diisocyanate (IPDI) and
   Q⁴ represents CH₂, CH₂—CH₂, CH₂ monoalkoxylate, CH₂—CH₂ monoalkoxylate, CH₂ polyalkoxylate or CH₂—CH₂ polyalkoxylate and
   a compound of formula (II):

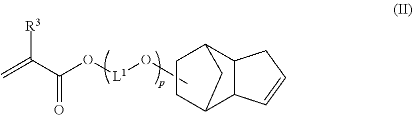

wherein:
   R³ independently represents H or CH₃,
   L¹ independently represents a straight or branched C₁-C₂₀-alkylene group, preferably an ethylene group or a propylene group and
   p independently represents 0 or an integer ranging from 1 to 30, for example from 1 to 20, particularly from 1 to 15, in particular from 1 to 10.

Monomer (a5) can also be chosen among di(meth)acrylates such as polyalkylene glycol di(meth)acrylate, in particular polypropylene glycol di(meth)acrylate, ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, but also 2,2′-bis(4-(acryloxy-propyloxyphenyl))propane, 2,2′-bis(4-(acryloxydiethoxy-phenyl))propane and zinc acrylate; tri(meth)acrylate compounds such as trimethylolpropane tri(meth)acrylate and ethoxylated trimethylolpropane tri(meth)acrylate, trimethylolethane tri(meth)acrylate, pentaerythritol tri(meth)acrylate and tetramethylolmethane tri(meth)acrylate; tetra(meth)acrylate compounds such as di-trimethylolpropane tetra(meth)acrylate, tetramethylolmethane tetra(meth)acrylate and pentaerythritol tetra(meth)acrylate; hexa(meth)acrylate compounds such as dipentaerythritol hexa(meth)acrylate; penta(meth)acrylate compounds such as dipentaerythritol penta(meth)acrylate; allyl compounds such as allyl (meth)acrylate, diallyl phthalate, diallyl itaconate, diallyl fumarate, diallyl maleate; polyallyl sucrose ethers with from 2 to 8 groups per molecule, pentaerythritol polyallyl ethers such as pentaerythritol diallyl ether, pentaerythritol triallyl ether and pentaerythritol tetraallyl ether; trimethylolpropane polyallyl ethers such as trimethylolpropane diallyl ether (TMPDAE) and trimethylolpropane triallyl ether. Other polyunsaturated compounds include divinyl glycol, divinyl benzene, divinylcyclohexyl and methylenebisacrylamide. Monomer (a5) can also be prepared by an esterification reaction of a polyol with an unsaturated anhydride such as acrylic anhydride, methacrylic anhydride, maleic anhydride or itaconic anhydride. To obtain monomer (a5), it is also possible to use compounds chosen among polyhaloalkanols such as 1,3-dichloroisopropanol and 1,3-dibromoisopropanol; hal oepoxyalkanes such as epi chlorohydrin, epibromohydrin, 2-methyl epichlorohydrin and epiiodohydrin; polyglycidyl ethers such as 1,4-butanediol diglycidyl ether, glycerin-1,3-diglycidyl ether, ethylene glycol diglycidyl ether, propylene glycol diglycidyl ether, diethylene glycol diglycidyl ether, neopentyl glycol diglycidyl ether, polypropylene glycol diglycidyl ether, bisphenol A-epichlorohydrin epoxy resin and mixtures thereof.

Monomer (a5) can also be chosen among the trifunctional cross-linking agents. This can be in particular trimethylolpropane tri(meth)acrylate (TMPTA) or trimethylolpropane ethoxylate tri(meth)acrylate (such as TMPTA 3EO).

Monomer (a5) can also be chosen among trimethylolpropane tri(meth)acrylate, ethoxylated trimethylolpropane tri(meth)acrylate, ethylene glycol di(meth)acrylate, methylene(bis)acrylamide, diallyl phthalate, diallyl maleate, trimethylolpropane diallyl ether (TMPDAE) and mixtures thereof.

Monomer (a5) can also be a mixture of two separate monomers, such as EGDCPEA (ethylene glycol dicyclopentenyl ether acrylate) and TMPTA or EGDCPEA and TMPTA 3EO or even EGDCPEMA (ethylene glycol dicyclopentenyl ether methacrylate) and TMPTA or EGDCPEMA and TMPTA 3EO.

According to the invention, monomer (a5) is preferably chosen among a compound of formula (I), a compound of formula (II), trimethylolpropane trimethacrylate, trimethylolpropane triacrylate, ethoxylated trimethylolpropane trimethacrylate, ethoxylated trimethylolpropane triacrylate, ethylene glycol dimethacrylate, ethylene glycol diacrylate, methylene(bis)acrylamide, diallylphthalate, diallylmaleate, trimethylolpropane diallyl ether (TMPDAE) and mixtures thereof.

Also advantageously, the polymerisation reaction can use less than 5 mol %, preferably from 0.01 to 4 mol %, particularly from 0.02 to 4 mol % or from 0.02 to 2 mol %, in particular from 0.02 to 1 mol %, of monomer (a5) relative to the total molar amount of monomers.

According to the invention, the associative monomer (a6) can be chosen among a compound of formula (III):

R¹-(EO)ₘ(PO)ₙ—R² (III)
wherein:
   m and n, identical or different, independently represent 0 or an integer or decimal less than 150, m or n being different from 0,
   EO independently represents a CH₂CH₂O group,
   PO independently represents a group chosen among CH(CH₃)CH₂O and CH₂CH(CH₃)O,
   R¹ represents an acrylate group or a methacrylate group and
   R² represents a straight or branched C₆-C₄₀-alkyl group, a phenyl group, a polyphenyl group, preferably a straight or branched C₁₀-C₃₀-alkyl group, more preferentially a straight or branched C₁₂-C₂₂-alkyl group, or a group comprising 2 to 5 phenyls or a tristyrylphenyl group or a pentastyrylcumylphenyl group.

Also advantageously according to the invention, the polymerisation reaction can use less than 20 mol %, preferably from 0.05 to 20 mol %, particularly from 0.1 to 10 mol %, of monomer (a6) relative to the total molar amount of monomers.

Preferably, the polymer (P) is prepared from the sole monomers (a1) to (a5), particularly from combinations of the sole monomers (a1)-(a2), (a1)-(a2)-(a3), (a1)-(a2)-(a4), (a1)-(a2)-(a5), (a1)-(a2)-(a3)-(a4), (a1)-(a2)-(a3)-(a5), (a1)-(a2)-(a4)-(a5), (a1)-(a2)-(a3)-(a4)-(a5), in particular from combination of the sole monomers (a1)-(a2), (a1)-(a2)-(a3) and (a1)-(a2)-(a5). Also preferably, the polymer (P) is prepared from combinations of the sole monomers (a1)-(a2)-(a6) and (a1)-(a2)-(a5)-(a6).

More preferably, the polymer (P) is prepared from the sole monomers (a1) and (a2) or from the sole monomers (a1)-(a2)-(a3), (a1)-(a2)-(a5), (a1)-(a2)-(a6) and (a1)-(a2)-(a5)-(a6).

Advantageously according to the invention, the polymerisation reaction uses:
from 19.9 to 66 mol % or from 29.8 to 66 mol %, preferably from 29.8 to 53 mol % or from 29.8 to 49 mol %, of monomer (a1),
from 30 to 80 mol % or from 40 to 70 mol %, preferably from 45 to 70 mol % or from 50 to 70 mol %, of monomer (a2),
from 0.01 to 4 mol % or from 0.02 to 4 mol %, preferably from 0.02 to 2 mol % or from 0.02 to 1 mol %, of monomer (a5),
   relative to the total molar amount of these three monomers.

Also preferably, the polymer (P) is not a styrenic polymer. It is then prepared in the absence of any styrene.

Preferably according to the invention, the polymer (P) can be fully or partially neutralised, preferably by means of at least one compound chosen among NaOH, KOH, ammonium derivatives, ammonia, amine bases, for example triethanolamine, aminomethyl propanol, or 2-amino-2-methyl-propanol (AMP) and combinations thereof.

Also preferably according to the invention, the polymer (P) can be fully or partially coacervated, more preferentially:
by reducing the pH, for example by reducing the pH to a value of less than 6.5, in particular by means of an acid compound, in particular by means of least one organic or inorganic acid compound, in particular an acid compound chosen among phosphoric acid, citric acid, glucono-lactone, lactic acid, salicylic acid, glycolic acid, ascorbic acid, glutamic acid, hydrochloric acid, acetic acid, D-gluconic acid, sulphonic acid, methanesulphonic acid, benzimidazole sulphonic acid, tartaric acid, 4-aminobenzoic acid, benzoic acid, sorbic acid, phenylbenzimidazole sulphonic acid, benzylidene camphor sulphonic acid, terephthalylidene dicamphor sulphonic acid, kojic acid, hyaluronic acid or
by increasing the ionic strength, for example by adding at least one ionised compound or at least one salt, in particular NaCl, KCl, MgCl₂, CaCl₂, MgSO₄, CaSO₄ or by adding phenylbenzimidazole sulphonic acid (PBSA) or pyroglutamic acid sodium salt (NaPCA) salt or even by adding at least one ionised organic sun filter.

In the composition according to the invention, the amount of polymer (P) present in the hydrophilic phase can vary. Preferably, the composition according to the invention comprises from 0.4 to 9% by weight or from 0.4 to 8% by weight, preferably from 0.5 to 10% by weight or from 0.5 to 9% by weight or from 0.5 to 8% by weight or even from 0.6 to 10% by weight or from 0.6 to 9% by weight or from 0.6 to 8% by weight, of polymer (P) relative to the amount of hydrophilic phase.

Generally, the composition according to the invention comprises a supplementary amount of hydrophilic compound, for example from 90 to 99.6% by weight of hydrophilic compound relative to the amount of hydrophilic phase when the polymer (P) is present in an amount of from 0.4 to 10% by weight.

In addition to the lipophilic and hydrophilic phases, the composition according to the invention can comprise other substances, in particular other substances used in cosmetics. The polymer (P) used according to the invention is not a surface-active compound. A surface-active compound works by forming a monolayer at the water-to-oil interface that reduces the surface tension of the particles of lipophilic compound. This surface-active compound forms micelles. For this, a molecule of surface-active compound comprises two distinct portions: a lipophilic portion and a hydrophilic portion.

Advantageously according to the invention, the polymer (P) makes it possible to control the texture and stability of the emulsion according to the invention. It also allows the UV-stabilizing cosmetic composition according to the invention to remain on the surface of the skin after its application. When applied, the UV-stabilizing cosmetic composition according to the invention thus forms a thin layer of lipophilic phase on the surface of the skin. This property can be obtained in the absence of any film-forming agent.

Furthermore, the use of the UV-stabilizing cosmetic composition according to the invention allows the UV-stabilizing filters to be retained in the skin areas less likely to react with the UV-stabilizing agent. The polymer (P) used makes it possible to limit or prevent the penetration of the UV-stabilizing agent through a hydrophobic substrate, particularly through the skin barrier.

Advantageously, the composition according to the invention can be used in a formulation. The invention therefore also relates to a formulation comprising at least one UV-stabilizing cosmetic composition according to the invention. Preferably, the formulation according to the invention is chosen among a sunscreen formulation, a makeup formulation, a skin care formulation and a hair care formulation.

In addition to the UV-stabilizing cosmetic composition according to the invention and this formulation, the invention also relates to the preparation of the UV-stabilizing cosmetic composition according to the invention.

Thus, the invention provides a method of preparing a UV-stabilizing cosmetic composition in the form of an emulsion, prepared in the absence of any surface-active compounds, and comprising:
the preparation of a continuous hydrophilic phase comprising:
   from 90 to 99.6% by weight of the hydrophilic phase of at least one hydrophilic compound and
   from 0.4 to 10% by weight of the hydrophilic phase of at least one emulsifying polymer (P) prepared by at least one polymerisation reaction:
      (a1) of at least one anionic monomer comprising at least one polymerisable olefinic unsaturation and at least one carboxylic acid group or of one of its salts and
      (a2) of at least one C₁-C₇ ester of a compound derived from an acid chosen among acrylic acid, methacrylic acid, maleic acid, maleic anhydride, itaconic acid and crotonic acid,
the preparation of a lipophilic phase comprising at least one lipophilic compound and at least one UV-stabilizing agent chosen among an organic sun filter, mineral particles and combinations thereof, then
the addition under stirring and in the absence of any surface-active compound of the lipophilic phase in the hydrophilic phase.

Particularly advantageously according to the invention and due to the emulsifying nature of the polymer (P), the UV-stabilizing cosmetic composition is prepared in the absence of any surface-active compound usually used when preparing oil-in-water compositions in the prior art. The preparation of the composition according to the invention is carried out in the absence of any amphiphilic compound.

Preferably, the addition is performed at a temperature ranging from 10° C. to 90° C. or from 15° C. to 75° C. or at room temperature.

Also preferably, the preparation method according to the invention also comprises the final adjustment of the pH of the composition. Preferably, the pH is adjusted to a value ranging from 4 to 13 or from 4 to 12 or from 4 to 11. More preferentially, the pH is adjusted to a value ranging from 5 to 13 or from 5 to 12 or from 5 to 11. Much more preferentially, the pH is adjusted to a value ranging from 6 to 13 or from 6 to 12 or from 6 to 11.

When preparing the UV-stabilizing cosmetic composition according to the invention, the addition of the lipophilic phase in the hydrophilic phase is performed under stirring, preferably under stirring with a device producing a shear gradient ranging from 500 to 10,000 s⁻¹.

The particular, advantageous or preferred characteristics of the UV-stabilizing cosmetic composition according to the invention define preparation methods according to the invention which are also particular, advantageous or preferred.

Moreover, the invention also relates to the use of the UV-stabilizing cosmetic composition according to the invention or the use of the formulation according to the invention.

Preferably, the UV-stabilizing cosmetic composition according to the invention is intended to be applied to the skin. More preferably, the UV-stabilizing cosmetic composition according to the invention is applied from 1 to 6 times a day, particularly on the face and the parts of the body exposed to light or to radiation from the sun.

Particularly advantageously, the polymer (P) used according to the invention makes it possible to control the transfer of the UV-stabilizing agent into a hydrophobic substrate such as skin. Thus, when applying a cosmetic composition according to the invention, optionally followed by leaching, the UV-stabilizing agent in the composition does not penetrate the hydrophobic substrate.

The invention provides a method of controlling penetration into a hydrophobic substrate of a UV-stabilizing cosmetic composition in the form of an emulsion prepared in the absence of any surface-active compound, comprising:
the preparation of a UV-stabilizing cosmetic composition according to the invention then
the application of this composition to a hydrophobic substrate, optionally
the final leaching of the hydrophobic substrate.

Generally, when applied to a hydrophobic substrate a cosmetic composition is transferred into this hydrophobic substrate. The amount of composition remaining on the surface of the hydrophobic substrate is then reduced.

Due to the polymer (P) according to the invention, the cosmetic composition according to the invention has a high resistance to transfer into a hydrophobic substrate.

The invention also provides a method of controlling or reducing the transfer of a UV-stabilizing cosmetic composition in the form of an emulsion prepared in the absence of any surface-active compound, in a hydrophobic substrate comprising:
the preparation of a UV-stabilizing cosmetic composition according to the invention then
the application of this composition to a hydrophobic substrate, optionally
the final leaching of the hydrophobic substrate.

According to the invention, the particular, advantageous or preferred characteristics of the UV-stabilizing cosmetic composition according to the invention define methods of controlling the penetration or of controlling or reducing the transfer of a UV-stabilizing cosmetic composition according to the invention which are also particular, advantageous or preferred.

According to the invention, controlling the penetration of the UV-stabilizing agent is achieved using a method (M) comprising the following successive steps:
spreading the UV-stabilizing cosmetic composition on a hydrophobic substrate (parafilm) using a 200 μm wedge (Elcometer model 3540),
drying the substrate for 4 hours at room temperature,
manually cleaning the surface of the coated substrate using an aqueous solution containing 10% of liquid soap,
rinsing the surface of the substrate with water from the loop,
drying the substrate by lightly dabbing with a sheet of paper towel,
placing the substrate in a UV chamber (Datacolor Tru-Vue2),
photographing the substrate (Sony Cyber-shot DSC-HX20V device) and
analysing and processing the image using ImageJ software (2011).

The grayscale distribution of the pixels in the image corresponds to the amount of UV-stabilizing agent that has penetrated the hydrophobic substrate. “0” corresponds to black and to the maximum penetration, while “255” corresponds to white and to zero penetration of the substrate by the UV-stabilizing agent. A value V50 is determined that corresponds to the grey level of an analysed substrate area (540×720 pixels) in which 50% of the pixels are lighter and 50% of the pixels are darker, rounded to the nearest integer.

Thus, the invention provides a method of control or of reduction according to the invention in which the value V50 of penetration or of transfer of the UV-stabilizing cosmetic composition is less than 150 measured according to the method (M) according to the invention and explained in the examples.

The invention also provides a cosmetic treatment method using a UV-stabilizing cosmetic composition according to the invention or using a formulation according to the invention or using a composition prepared according to the method of preparing a UV-stabilizing cosmetic composition according to the invention. The cosmetic treatment method according to the invention comprises the application to a hydrophobic substrate, in particular the application to the skin, of this composition or of this formulation, optionally followed by the leaching of the hydrophobic substrate.

According to the invention, the particular, advantageous or preferred characteristics of the UV-stabilizing cosmetic composition according to the invention define cosmetic treatment methods according to the invention which are also particular, advantageous or preferred.

The following examples illustrate the various aspects of the invention.

### EXAMPLES

The following abbreviations are used:
MAA: Methacrylic Acid,
EA: Ethyl Acrylate,
AMPS: 2-acrylamido-2-methylpropanesulphonic acid,
DAP: Diallyl Phthalate,
Clariant Polyglykol B11/50: propylene oxide ethylene oxide monobutyl ether,
Huntsmann Empicol LXVN: sodium lauryl sulphate (SLS),
BASF Texapon NSO: ammonium laureth sulphate in 28% solution or ammonium lauryl ether sulphate in 28% solution (SLES),
Sodium persulphate (NH₄)₂S₂O₈, sodium metabisulphite Na₂S₂O₅,
Straight C₁₆-C₁₈-(EO)25-alkyl methacrylate (monomer (a6)).

### Example 1: Preparation of Polymers (P1), (P2), (P3) and (P4) According to the Invention According to a Semi-Batch Method

In a stirred 1 L reactor heated using an oil bath, mixture 1 is prepared by introducing deionised water and a sodium lauryl sulphate (SLS) solution or an aqueous solution containing 28% by mass of sodium lauryl ether sulphate (SLES), optionally propylene oxide ethylene oxide monobutyl ether (B11/50) and optionally monomer (a3), 2-acrylamido-2-methylpropane sulphonic acid (AMPS).

A mixture 2 is prepared in a beaker, called a monomer premixture, comprising in deionised water:
monomer (a1), methacrylic acid (MAA),
monomer (a2), ethyl acrylate (EA),
monomer (a5), diallyl phthalate,
optionally monomer (a3), AMPS,
optionally a 28% solution of sodium lauryl ether sulphate (SLES) or of sodium lauryl sulphate (SLS),
optionally an associative monomer (a6) of formula (III), straight C₁₆-C₁₈(EO)25-alkyl methacrylate,
optionally a transfer agent, n-dodecyl mercaptan.

This premixture is stirred to form a monomer mixture.

An initiator solution is prepared comprising ammonium persulphate and deionised water and optionally sodium metabisulphite. All reagents and amounts used are listed in Table 1.

For polymers (P1), (P2) and (P4) used according to the invention, the reactor is heated to 85° C.±1° C. and the initiator solution and monomer premixture are injected in parallel, over 2 hours. This mixture is baked for 30 min at 85° C.±1° C. and then cooled to room temperature.

For polymer (P3) used according to the invention, the reactor is heated to 74° C.±1° C., all of the initiator solution is injected as soon as the reactor has come to the set temperature and the monomer premixture is injected over 2 hours.

This mixture is baked for 30 min at 74° C.±1° C. and then cooled to room temperature.

The polymers according to the invention were prepared under these conditions by varying the monomer compositions of the monomer premixtures. The copolymer compositions obtained are shown in Table 1.

TABLE 1
Polymer

| Amount (g) | | P1 | P2 | P3 | P4 |
|---|---|---|---|---|---|
| Mixture 1 | Deionised water | 400 | 400 | 359.1 | 261.7 |
| | SLS | 2.60 | 0 | 2.7 | 3.8 |
| | SLES | 0 | 6.17 | 0 | 0 |
| | Polyglykol B11/50 | 1.10 | 0 | 0 | 0 |
| | AMPS (a3) | 0 | 0 | 4.5 | 0 |
| Premixture | Deionised water | 173.70 | 175.00 | 126.4 | 343.3 |
| | SLS | 1.81 | 0 | 0.9 | 3.8 |
| | SLES | 0 | 4.29 | 0 | 0 |
| | Polyglykol B11/50 | 1.04 | 0 | 0 | 0 |
| | MAA (a1) | 105.69 | 88.15 | 91.30 | 114.0 |
| | EA (a2) | 191.28 | 176.36 | 156.24 | 84.5 |
| | Diallyl phthalate (a5) | 2.75 | 0.88 | 0 | 0 |
| | Monomer (a6) | 0 | 22.96 | 0 | 29.5 |
| | N-dodecyl mercaptan | 0 | 0 | 0 | 0.9 |
| Initiator | Deionised water | 54.32 | 62.85 | 24.5 | 65.2 |
| | Ammonium persulphate | 0.58 | 0.41 | 0.88 | 0.60 |
| | Sodium metabisulphite | 0 | 0 | 0.08 | 0 |
| Composition | monomer (a1) (mol %) | 38.98 | 32.51 | 40.13 | 60.5 |
| | Monomer (a2) (mol %) | 60.67 | 55.93 | 59.05 | 38.55 |
| | Monomer (a3) (mol %) | 0 | 0 | 0.82 | 0 |
| | Monomer (a5) (mol %) | 0.35 | 0.11 | 0 | 0 |
| | Monomer (a6) (mol %) | 0 | 0.52 | 0 | 0.95 |
| Characteristics | Final solids content (% by weight) | 30 | 29 | 30 | 30 |
| | Particle size (nm) | 74 | 75 | 120 | 100 |

### Example 2: Preparation and Evaluation of UV-Stabilizing Cosmetic Compositions (C1 to C4) According to the Invention and Comparative UV-Stabilizing Cosmetic Compositions (CC1 to CC3)

The hydrophilic phase is prepared in a beaker by stirring, using an lka or Rayneri motorised paddle stirrer, deionised water, butyl ene glycol, di sodium ethylenediaminetetraaceticacid (Na₂EDTA), phenylbenzimidazole sulphonic acid (PBSA, Parsol HS) and a polymer (P1) or (P2) or (P3) or (P4) according to the invention or a surface-active compound alone or in combination with a rheology-modifying polymer. The pH is adjusted to 7±0.5 by adding a 20% sodium hydroxide solution in water.

The hydrophilic phase of the compositions according to the invention and the comparative compositions is prepared under stirring at 700-1,000 rpm (except composition CC1 at 1,600-2,000 rpm).

The rheology-modifying polymer in the comparative examples (CC2) and (CC3) is Rheostyl UP (Coatex— INCI: acrylate copolymer).

Comparative compositions (CC1), (CC2) and (CC3) comprise a surface-active compound (Seppic, Oramix CG 110—INCI: Caprylyl/Capryl Glucoside).

The lipophilic compounds of the lipophilic phase are:
Cetiol B (INCI: dibutyl adipate),
Dicaprylyl carbonate (INCI: dicaprylyl carbonate),
Parsol 340 (INCI: octocrylene),
Parsol MCX (INCI: ethylhexyl cinnamate) and
Parsol 1789 (INCI: avobenzone).

The other compounds then added are:
Parsol 50 TX AB (INCI: titanium dioxide with silica and dimethicone
Bioxan E 1,000 UI (INCI: tocopheryl acetate),
Phenoxyethanol (INCI: phenoxyethanol) and
Coconut fragrance (INCI: fragrance).

The ingredients and amounts are listed in Tables 2 and 3 along with the characteristics and properties of the UV-stabilizing cosmetic compositions obtained.

Brookfield viscosity is measured at 6 rpm and at 25° C.

To evaluate the transfer of the UV-stabilizing cosmetic composition comprising UV-stabilizing filters to a hydrophobic substrate, a carrier is used with similar but more restrictive characteristics than those of the skin. Indeed, the hydrophobic substrate used is more hydrophobic than the skin and therefore will allow for easier penetration of the UV-stabilizing agents. Moreover, and contrary to the skin, the hydrophobic substrate used is not porous, so it will enable a more difficult penetration of the UV-stabilizing agents.

The composition is spread on a parafilm (Bemis flexible packaging) using a 200 μm wedge (Elcometer model 3540) and allowed to dry for 4 hours at room temperature. Then, the surface of the coated parafilm is manually cleaned with an aqueous solution containing 10% of liquid soap, then flushed with water from the loop and dried by lightly dabbing with a paper towel. The parafilm is then placed in a UV chamber (Datacolor Tru-Vue2) and a photo of the parafilm is taken (Sony Cyber-shot DSC-HX20V device) and analysed using ImageJ software (2011).

The darker the parafilm obtained, the greater the transfer of the UV-stabilizing cosmetic composition and of the UV-stabilizing agent. Image processing makes it possible to identify the grayscale distribution of the pixels in the image (from 0 which corresponds to black to 255 which corresponds to white). The V50 value corresponds to the grey level for which one half of the pixels are lighter and the other half are darker. The results obtained are shown in Tables 2 and 3.

TABLE 2

| Composition according to the invention (g) | C1 | C2 | C3 | C4 |
|---|---|---|---|---|
| Hydrophilic phase: deionised water | 66.55 | 66.55 | 66.55 | 66.55 |
| Hydrophilic phase: butylene glycol | 3 | 3 | 3 | 3 |
| Hydrophilic phase: Na₂EDTA | 0.05 | 0.05 | 0.05 | 0.05 |
| Hydrophilic phase: Parsol HS | 0.5 | 0.5 | 0.5 | 0.5 |
| Hydrophilic phase: P1 at 30% in water | 4 | 0 | 0 | 0 |
| Hydrophilic phase: P2 at 29% in water | 0 | 4 | 0 | 0 |
| Hydrophilic phase: P3 at 30% in water | 0 | 0 | 4 | 0 |
| Hydrophilic phase: P4 at 30% in water | 0 | 0 | 0 | 4 |
| Lipophilic phase: Cetiol B | 6 | 6 | 6 | 6 |
| Lipophilic phase: Dicaprylyl carbonate | 1 | 1 | 1 | 1 |
| Lipophilic phase: Parsol 340 | 6 | 6 | 6 | 6 |
| Lipophilic phase: Parsol MCX | 5 | 5 | 5 | 5 |
| Lipophilic phase: Parsol 1789 | 2.5 | 2.5 | 2.5 | 2.5 |
| Other Ingredients: Parsol 50TX AB | 4 | 4 | 4 | 4 |
| Other Ingredients: Bioxan E | 0.2 | 0.2 | 0.2 | 0.2 |
| Other Ingredients: Phenoxyethanol | 1 | 1 | 1 | 1 |
| Other Ingredients: Fragrance | 0.2 | 0.2 | 0.2 | 0.2 |
| viscosity (mPa · s) | 5,570 | 11,900 | 20,400 | 20,800 |
| pH | 7.0 | 6.9 | 7.0 | 6.8 |
| V50 | 153 | 156 | 153 | 151 |

TABLE 3

| Comparative composition (g) | CC1 | CC2 | CC3 |
|---|---|---|---|
| Hydrophilic phase: deionised water | 66.55 | 63.05 | 66.55 |
| Hydrophilic phase: butylene glycol | 3 | 3 | 3 |
| Hydrophilic phase: Na₂EDTA | 0.05 | 0.05 | 0.05 |
| Hydrophilic phase: Parsol HS | 0.5 | 0.5 | 0.5 |
| Hydrophilic phase: Oramix CG 110- | 4 | 4 | 0.5 |
| 60% in water | | | |
| Hydrophilic phase: 30% Rheostyl UP | 0 | 3.5 | 3.5 |
| in water | | | |
| Lipophilic phase: Cetiol B | 6 | 6 | 6 |
| Lipophilic phase: dicaprylyl carbonate | 1 | 1 | 1 |
| Lipophilic phase: Parsol 340 | 6 | 6 | 6 |
| Lipophilic phase: Parsol MCX | 5 | 5 | 5 |
| Lipophilic phase: Parsol 1789 | 2.5 | 2.5 | 2.5 |
| Other Ingredients: Parsol 50TX AB | 4 | 4 | 4 |
| Other Ingredients: Bioxan E | 0.2 | 0.2 | 0.2 |
| Other Ingredients: Phenoxyethanol | 1 | 1 | 1 |
| Other ingredients: Fragrance | 0.2 | 0.2 | 0.2 |
| Viscosity (mPa · s) | 6,900 | <100 | 10,000 |
| pH | 7.5 | 7.0 | 6.9 |
| V50 | 148 | 147 | 149 |

Quite surprisingly, it can be seen that the UV-stabilizing cosmetic compositions according to the invention provide a better resistance to penetration of the UV-stabilizing agents in the hydrophobic substrate than the comparative compositions. It can be seen that this resistance to penetration is not related to the associativity of the polymer or that it is not related to the cross-linking of the polymer.

The highest resistance to penetration of the UV-stabilizing agents in the hydrophobic substrate of the compositions according to the invention depends neither on the viscosity of the composition nor on its suspensive properties. It does not depend on the use of an associative monomer when preparing the polymer (P) used.

Thus, polymers (P1), (P2), (P3) and (P4) according to the invention make it possible to prepare UV-stabilizing cosmetic compositions that significantly limit transfers of UV-stabilizing agents to hydrophobic substrates. Furthermore, the use of a surface-active compound in the comparative cosmetic compositions does not make it possible to obtain a UV-stabilizing cosmetic composition that significantly limits transfers of lipophilic UV-stabilizing agents to hydrophobic substrates.

The UV-stabilizing cosmetic compositions according to the invention therefore offer dual protection: they protect against UV radiation and they significantly limit the transfers of UV-stabilizing agents.

1. A UV-stabilizing cosmetic composition in the form of an emulsion prepared in the absence of any surface-active compound, the composition comprising, based on total composition weight:
1 to 75 wt % of lipophilic phase particles comprising a lipophilic compound and a UV-stabilizing agent comprising an organic sun filter and/or a mineral particle,
wherein the lipophilic phase particles are dispersed in:
25 to 99 wt. % of a continuous hydrophilic phase comprising, based on total hydrophilic phase weight:
a hydrophilic compound in a range of from 90 to 99.6 wt. %, and
an emulsifying polymer (P) in a range of from 0.4 to 10 wt. %,
wherein the emulsifying polymer (P) is prepared by a process comprising polymerizing:
(a1) an anionic monomer comprising a polymerizable olefinic unsaturation and a carboxylic acid group, optionally in salt form; and
(a2) a C₁-C₇ ester of a compound derived from acrylic acid, methacrylic acid, maleic acid, maleic anhydride, itaconic acid, or crotonic acid.

2. The composition of claim 1, comprising from 1 to 70 wt. % of dispersed lipophilic phase, relative to total continuous hydrophilic phase and dispersed lipophilic phase weight.

3. The composition according to one of claim 1, wherein the lipophilic phase comprises a lipophilic compound comprising a fatty acid, fatty alcohol, ester, butter, wax, oil, terpene, polyterpene, phytosterol, silicone oil, and/or fluorinated oil.

4. The composition of claim 1, wherein the UV-stabilizing agent comprises:
a titanium dioxide particle, zinc oxide particle, coated mineral particle, and/or a lipophilic UV-stabilizing agent.

5. The composition of claim 1, wherein the hydrophilic compound comprises water and optionally further comprises a hydrophilic UV-stabilizing agent, glycerol, polyglycerol, glycerol, and/or a moistener.

6. The composition of claim 1, having
a pH greater than 4, or
a pH less than 13, or
a pH in a range of from 4 to 13.

7. The composition of claim 1, wherein
the anionic monomer (a1) comprises acrylic acid, methacrylic acid, maleic acid, maleic anhydride, itaconic acid, crotonic acid, an acrylic acid salt, a methacrylic acid salt, a maleic acid salt, a maleic anhydride salt, an itaconic acid salt, a crotonic acid salt, or a combination of two or more of any of these, or
wherein the C₁-C₇ ester (a2) is a C₁-C₆ ester, or
wherein
the polymerization reaction uses, relative to total monomer (a1) and (a2) moles:
the anionic monomer (a1) in a range of from 20 to 75 mol. %, and
the C₁-C₇ ester (a2) in a range of from 25 to 80 mol.

8. The composition of claim 1, wherein the hydrophilic phase comprises the emulsifying polymer (P) in a range of from 0.4 to 9 wt. % relative to the hydrophilic phase weight.

9. The composition of claim 1, wherein the process for preparing the emulsifying polymer (P) further comprises polymerizing:
(a3) a third compound comprising 2-acrylamido-2-methylpropane sulphonic acid, ethoxymethacrylate sulphonic acid, sodium methallyl sulphonate, styrene sulphonate, hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, or a mixture thereof, optionally comprising one or more salts thereof, or
(a4) a fourth compound comprising hydroxyethyl-acrylate, hydroxypropyl-acrylate, hydroxyethylhexyl-acrylate, hydroxyethyl-methacrylate, hydroxypropyl-methacrylate, hydroxyethylhexyl-methacrylate, or a mixture thereof, or
(a5) a cross-linked monomer or a fifth monomer comprising at least two olefinic unsaturations, or
(a6) an associative monomer, preferably chosen among a optionally of formula (III):
R¹-(EO)ₘ-(PO)ₙ—R² (III),
wherein
m and n are independently 0 or an integer or decimal less than 150, wherein m or n is not 0,
EO is a CH₂CH₂O group,
PO is independently CH(CH₃)CH₂O or CH₂CH(CH₃)O,
R¹ is an acrylate group or a methacrylate group, and
R² is a straight C₆-C₄₀-alkyl group, a branched C₆-C₄₀-alkyl group, a phenyl group, or a polyphenyl group.

10. The composition of claim 1, wherein the emulsifying polymer (P) is at least partially neutralized,
wherein the emulsifying polymer (P) is at least partially coacervated.

11. A a sunscreen formulation, a makeup formulation, a skin care formulation and a hair care formulation, comprising:
a UV-stabilizing cosmetic composition of claim 1.

12. A method of preparing a UV-stabilizing cosmetic composition in the form of an emulsion, prepared in the absence of any surface-active compound, the method comprising:
preparing a continuous hydrophilic phase comprising, relative to total hydrophilic phase weight:
a hydrophilic compound in a range of from 90 to 99.6 wt. %; and
a emulsifying polymer (P) in a range of from 0.4 to 10 wt. %,
wherein the emulsifying polymer (P) is prepared by a process comprising polymerizing:
(a1) a anionic monomer comprising a polymerizable olefinic unsaturation and a carboxylic acid group or salt thereof; and
(a2) 1 C₁-C₇ ester of a compound derived from an acid comprising acrylic acid, methacrylic acid, maleic acid, maleic anhydride, itaconic acid and/or crotonic acid;
preparing of a lipophilic phase comprising a lipophilic compound and a UV-stabilizing agent comprising an organic sun filter and/or a mineral particle; then
adding, under stirring and in the absence of any surface-active compound, the lipophilic phase in the hydrophilic phase.

13. The method of claim 12, further comprising:
adjusting a final pH of the composition, or
wherein the adding is performed at a temperature in a range of from 10° C. to 90° C.

14. A method of preparing, the UV-stabilizing cosmetic of claim 1 in the form of an emulsion, the method comprising:
preparing the continuous hydrophilic phase;
preparing the lipophilic phase; then
adding, under stirring and in the absence of any surface-active compound, the lipophilic phase in the hydrophilic phase.

15. A method of controlling penetration into a hydrophobic substrate of a UV-stabilizing cosmetic composition in the form of an emulsion prepared in the absence of any surface-active compound, the method comprising:
preparing the UV-stabilizing cosmetic composition of claim 1; then
applying the UV-stabilizing cosmetic composition to a hydrophobic substrate; and, optionally,
finally leaching the hydrophobic substrate.

16. A method of controlling or reducing the transfer of a UV-stabilizing cosmetic composition in the form of an emulsion prepared in the absence of any surface-active compound, in a hydrophobic substrate, the method comprising:
preparing the UV-stabilizing cosmetic composition of claim 1; then
applying the UV-stabilizing cosmetic composition to a hydrophobic substrate; and, optionally, finally leaching the hydrophobic substrate.

17. The method of claim 15, having a V50 value of penetration or of transfer of the UV-stabilizing cosmetic composition of less than 150, measured by a method (M) involving:
spreading the UV-stabilizing cosmetic composition on a parafilm substrate using a 200 μm an Elcometer model 3540 wedge, to obtain a coated substrate;
drying the coated substrate for 4 hours at room temperature, to obtain a dried coated substrate;
manually cleaning a surface of the dried coated substrate using an aqueous solution containing 10% of liquid soap, to obtain a cleaned coated substrate;
rinsing the surface of the cleaned coated substrate with water, to obtain a rinsed substrate;
drying the rinsed substrate by dabbing with a sheet of paper towel, to obtain a dabbed substrate;
placing the dabbed substrate in a Datacolor Tru-Vue2 UV chamber;
photographing the dabbed substrate with a Sony Cyber-shot DSC-HX20V device, to obtain an image; and
analyzing and processing the image using 2011 ImageJ software, wherein a grayscale distribution of pixels in the image corresponds to an amount of UV-stabilizing agent that has penetrated the hydrophobic substrate, with 0 corresponding to black and to maximum penetration, and 255 corresponding to white and no penetration of the substrate by the UV-stabilizing agent.

18. A cosmetic treatment method, comprising:
applying the UV-stabilizing cosmetic composition of claim 1 onto a hydrophobic substrate, optionally followed by the leaching of the hydrophobic substrate.
